(19) United States
(12) Patent Application Publication
(10) Pub. No.: US 2023/0218189 A1
(43) Pub. Date: Jul. 13, 2023

(54) CLASSIFICATION OF RADIO FREQUENCY SCATTERING DATA

(71) Applicant: MEDFIELD DIAGNOSTICS AB, Göteborg (SE)

(72) Inventors: Mikael PERSSON, Alingsås (SE); Andreas FHAGER, Ytterby (SE); Tomas MCKELVEY, Alingsås (SE); Harald JACOBSSON, Göteborg (SE); Rikard VINGE, Hisings Backa (SE)

(21) Appl. No.: 18/009,154

(22) PCT Filed: Jun. 4, 2021
(86) PCT No.: PCT/EP2021/064979

§ 371 Date: Dec. 8, 2022

(30) Foreign Application Priority Data
Jun. 9, 2020 (SE) 2050674-7

(51) Int. Cl. A61B 5/0507; A61B 5/00; G16H 50/20

(52) U.S. Cl. CPC A61B 5/0507; A61B 5/7267; G16H 50/20

(57) ABSTRACT

The embodiments herein relate to a system and method for detecting an internal object in a body under test. The system comprises at least one antenna which are adapted to be positioned around the body under test. The system is adapted to transmit radio frequency signal(s) into the body under test which are reflected and/or scattered from the internal object. The system is adapted to receive the reflected and/or scattered radio frequency signal(s) and use a method of classification S1-S7 to determine the presence of an internal object. The system is adapted to detect the internal object or a change in an already detected internal object when there is a difference between the received radio frequency signals. The difference is related to the different dielectric properties between the internal object and the body under test.

### TECHNICAL FIELD

This disclosure relates to classification of measured data into classes. Example embodiments presented herein relate to the collection and analysis of radio frequency signal scattering data.

### BACKGROUND

In many application areas there is a need for detecting and probing the interior of an object or a body under test. Invasive methods where the object is cut open and physically investigated is perhaps the most straight forward method for assessing the interior of a body or object. In many cases however non-invasive and non-destructive methods are preferred or even necessary in order not to destroy the body under test. Many different non-invasive systems and methods exist on the market, some examples include methods based on X-rays, ultrasound, magnetic resonance, and a large range of other techniques are available. In any given application, the most suitable detection method is determined for example by considering the physical properties of the object under investigation and the type of internal properties that are subject to investigation. Other factors such as cost, size of equipment, and time for investigation must also be accounted for. Radio frequency-based methods are particularly appealing due to its specific interaction with matter and that the technology is developing fast and that components rapidly decrease in price and size. This enables sensitive and competitive detection and diagnostic applications, that has the potential to outperform more conventional techniques in many different areas.

### DESCRIPTION OF RELATED ART

EP2020915B1 describes a method and a system to reconstruct images from radio frequency signal scattering data.

EP2032030B1 describes a device, method, and system for monitoring the status of an internal part of a body using an electromagnetic transceiver operating in the microwave regime; radio frequency signal scattering data in from of time domain pulses are analysed to determine the location of the surface of the body (e.g. skin) and thereby enable compensation for movements.

EP2457195 B1 describes a device for determining an internal condition of a subject by analysis of an enclosed volume, by using a particular statistical classification algorithm based on training data.

U.S. Pat. No. 7,226,415 B2 describes an apparatus for detecting blood flow based on the differences in dielectric properties of tissue.

U.S. Pat. No. 6,4547,11 B1 relates to a haemorrhage detector. It describes an antenna array including matching medium between antennas and the skin, as well as damping material between antennas. The detection algorithm is based on analysing received time domain pulses and detecting changes in the received pulses due to haemorrhages.

U.S. Pat. No. 7,122,012 B2 describes a method of detecting a change in the level of fluid in tissue. The analysis is based on comparing the measurements with reference measurements on a target without the liquid present. The presence of fluid is based on differences between a base line signal and a measured signal.

U.S. Pat. No. 9,072,449 B2 discloses a system for wearable/man-portable electromagnetic tomographic imaging which includes a wearable/man-portable boundary apparatus adapted to receive a biological object within, a position determination system, electromagnetic transmitting/receiving hardware, and a hub computer system.

U.S. Pat. No. 9,414,749 B2 discloses an electromagnetic tomography system for gathering measurement data pertaining to a human head which includes an image chamber unit, a control system, and a housing.

US20150342472A1 discloses a method of assessing status of a biological tissue that includes transmitting an electromagnetic signal, via a probe, into a biological tissue. The electromagnetic signal is received after being scattered/reflected on its way through tissue. Blood flow information pertaining to the biological tissue is provided, and the received signal is analyzed based at least upon the provided blood flow information and upon knowledge of electromagnetic signal differences in normal and abnormal tissue.

### SUMMARY

An objective of the present disclosure is to provide improved measurement devices, systems and methods for classifying measurement data obtained via a microwave transceiver into classes. Some examples of the disclosure relate to systems and methods for providing improved detection of an internal object inside a body under test. Other examples relate to systems and methods for providing information whether an internal object is present or not present inside the body under test.

This objective is obtained by a method for classifying measurement data into one or more classes. The method comprises obtaining training data, determining a subspace base for each class out of the one or more classes based on the training data, determining principal angles between each pair of subspace bases, determining a component energy for each dimension in each subspace, determining a reduced dimension subspace for each class by discarding subspace dimensions based on respective principal angle and component energy, and classifying the measurement data into the one or more classes based on the reduced dimension subspaces.

The proposed classifier does not only use principal components, eigenvalues, singular values, or principal angles to truncate the subspace, but a combination of these. The principal components with the highest energy carry information on which constituents of the subspaces that carry the majority of the information for each individual class, while the principal angles contain information on the similarities between the subspaces. Thus, by combining the knowledge from the two, it is possible to construct subspaces that carry more information while achieving high separation between the class subspaces.

When applying radio frequency signal scattering data towards the detection and diagnostics, it should be appreciated that the number of dimensions of the measured data may exceed the number of training samples available. This problem is alleviated by the techniques proposed herein, due to the construction of reduced dimension subspaces.

According to aspects, the method comprises configuring an energy level E. Determining the reduced dimension subspace for each class then comprises discarding subspace dimensions while maintaining an energy level per subspace above the configured energy level E. This way, it is ensured that a certain amount of total energy is maintained in the classes after discarding components, which improves robustness of the proposed methods.

According to aspects, determining a component energy comprises rotating the respective subspace. The rotation is an efficient way to determine principal angles.

According to aspects, obtaining training data comprises normalizing the training data.

According to aspects, obtaining training data comprises standardizing the training data.

Consequently, the proposed classifier and associated techniques are compatible with a wide range of measurement data sets, comprising both normalized and/or standardized data sets, which is an advantage.

According to aspects, the classifying comprises obtaining a measurement data set, determining a distance between the measurement data set and at least one of the reduced dimension subspaces corresponding to the one or more classes, and associating the measurement data set with at least one class based on the determined distance. This way a likelihood of the measurement data being related to a certain class can be quantified.

There is also disclosed herein a diagnostic system or apparatus configured to detect presence of an object and/or configured to detect changes in properties of an object, comprised in an enclosing volume, where the object is associated with a dielectric property different from that of the volume. The disclosed diagnostic system comprises an analysis unit configured to perform one or more of the methods described herein.

The described techniques find applications in many different and diverse areas, ranging from medical diagnosis to industrial applications such as wood-processing industries and others.

Embodiments herein afford many additional advantages, of which a non-exhaustive list of examples follows:

One advantage with the embodiments herein is that the method is particularly suitable when the amount of training data is small, i.e. when the number of objects used for training is lower than the dimensionality of one measurement. A situation that exemplifies this could for example be a case where 100 objects have been measured for the training of the classification algorithm, and where the measurement on each of these objects were made at 1000 different frequency points.

Prior art relating to training of a classification algorithm for diagnosing or detection of data commonly only use one of the following features to truncate the subspaces and thus as a basis for separating classes: principal components, eigenvalues, singular values, or principal angles. The separation of classes is the most important feature for the performance of the classifier, where better separation between subspaces leads to a more accurate detection. The subspaces are created based on training data. After the training has been made and the subspaces created the classification can be based on determining a distance measure between the subspaces and single data point associated with a measurement on a body under test. The subspace closest to the data point is in such classifier used to determine which class the measured data point belongs to. To exemplify, one subspace representing the presence of an internal object inside a body under test, and one subspace representing the non-presence of an internal object inside a body could be used to do determine if an internal object is presence or not inside a body under test. The principal components with the highest energy carry information on which constituents of the subspaces that carry the majority of the information for each individual class, while the principal angles contain information on the similarities between the subspaces. The embodiments herein relate to a method where the information from the two features is used in an optimal and efficient way such that the subspaces carry more information while achieving high separation between the class subspaces compared to when using the features separately.

A further advantage of the embodiments herein is that in principle, all conditions inside a body under test where there is a dielectric contrast with respect to the surrounding dielectric properties and/or where the level of dielectric contrast changes over time and/or where the size of the region constituting the dielectric contrast changes over time may be detected.

Another advantage of the embodiments herein is that they provide solutions for handling radio frequency signal scattering data and provides a more reliable result for interpretation of the data and a more reliable diagnosis of the internal properties, i.e. the internal object, of the body under test.

Another advantage is that the self-learning approach will make the classification algorithm perform better and better the more samples that are included in the training data. Every measurement that is made after an initial training phase therefor has the potential to improve future classification as it can be added to the training data when an independent verification confirms the presence or absence of the internal object.

The embodiments herein are not limited to the features and advantages mentioned above. A person skilled in the art will recognize additional features and advantages upon reading the following detailed description.

### BRIEF DESCRIPTION OF THE DRAWINGS

FIG. 1 is a schematic diagram illustrating an example measurement device or diagnostics system.

FIG. 2 is a schematic diagram illustrating another example measurement device or diagnostics system.

FIG. 3 is a schematic diagram illustrating training and use of the classification algorithm.

FIG. 4 is a flow chart illustrating methods.

FIG. 5 is a schematic diagram illustrating yet another example measurement device or diagnostics system.

FIG. 6 shows an overview of example modules comprised in processing circuitry.

FIG. 7 schematically illustrates a control unit or analyzer.

FIG. 8 shows a computer program product.

### DETAILED DESCRIPTION

The embodiments herein relate to detection of an internal object 100 inside of a body under test 103, where the internal object 100 is associated with a dielectric property different from that of the body under test 103, and more specifically to detection of an internal object 100 by means of a self-learning classification algorithm S1-S7. This may also be referred to as detection of one or more dielectric targets, with certain properties, such as size, shape, position, dielectric parameters, etc. that is immersed inside another dielectric medium. A further description of the embodiments herein is that they relate to interrogating the interior of a body under test 103 and to detect the presence, or occurrence of variations in the properties of, one or more internal objects 100 with different dielectric property than that of the body under test 103.

The detection or interrogation of the body under test 103 is performed using radio frequency signals, such as radio frequency signals in the microwave regime. As an example, the radio frequency signals may comprise signals in the frequency range 100 MHz to 10 GHz or more. Herein, the terms microwave signals and radio frequency signals will be used interchangeably. It is thus appreciated that the term microwave signal is given a broad interpretation herein, and is not limited to, e.g., a specific frequency band or the like.

A dielectric property of an object may, e.g., be associated with a dielectric constant of the object. The dielectric constant is the ratio of the permittivity of a substance to the permittivity of free space. The dielectric property of a substance or object may also be associated with a permittivity and a conductivity of a substance and thereby the dielectric constant is represented in form of a complex number. The definition and implication of dielectric properties, represented as a permittivity, conductivity or a complex dielectric parameter is well known by a person skilled in the art of microwave theory and practice, and will therefore not be discussed in detail herein.

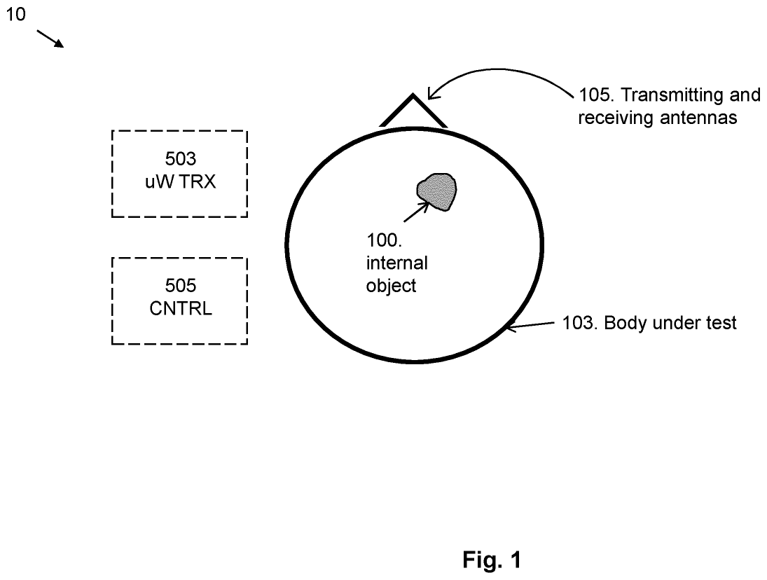

FIG. 1 schematically illustrates an example measurement device 10 or diagnostics system. The two terms diagnostic system and measurement device will be used interchangeably herein.

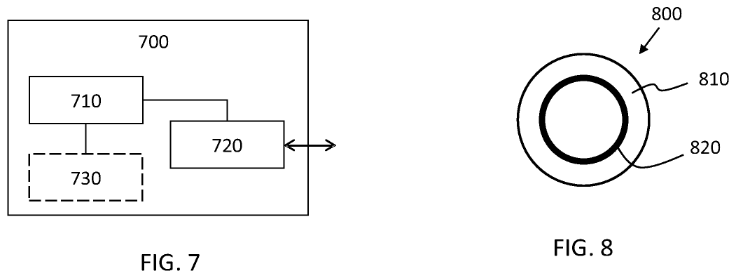

The measurement device 10 comprises at least one transmitting antenna, at least one receiving antenna, a microwave transceiver unit (uW TRX) 503 connected to the at least one transmitting antenna and to the at least one receiving antenna, and a control unit (CNTRL) 505 or analyzer connected to the microwave transceiver unit. The microwave transceiver 503 and the control unit 505 are only schematically illustrated in FIG. 1. The control unit comprises processing circuitry arranged to classify measurement data obtained via the microwave transceiver unit 503 into one or more pre-determined classes. The control unit 505 will be discussed in more detail below in connection to FIG. 7.

At least part of the present disclosure relates to this measurement device 10 or diagnostic system configured to detect presence of an internal object 100 or configured to detect changes in properties of an internal object 100, comprised in an enclosing volume or body under test 103, where the internal object 100 is associated with a dielectric property different from that of the volume or body under test 103. This detection is part of the classification. For instance, two classes can be defined: ‘foreign object present’ and ‘foreign object not present’. If a given set of measurement data is then classified into the ‘foreign object present’ class, a foreign object has been detected. Thus, it is appreciated that a classification operation may be seen as a detection operation, and vice versa.

The diagnostic system comprises at least one antenna 105 which is adapted to be positioned at locations around the body under test 103. It is appreciated that the body under test 103 may be a patient or part of a patient, i.e., a human or animal, or may be some other body under test 103, such as a material of wood, a log or tree. Materials such as construction material, soil, rocks, water, and other material and substances could also constitute the body under test 103.

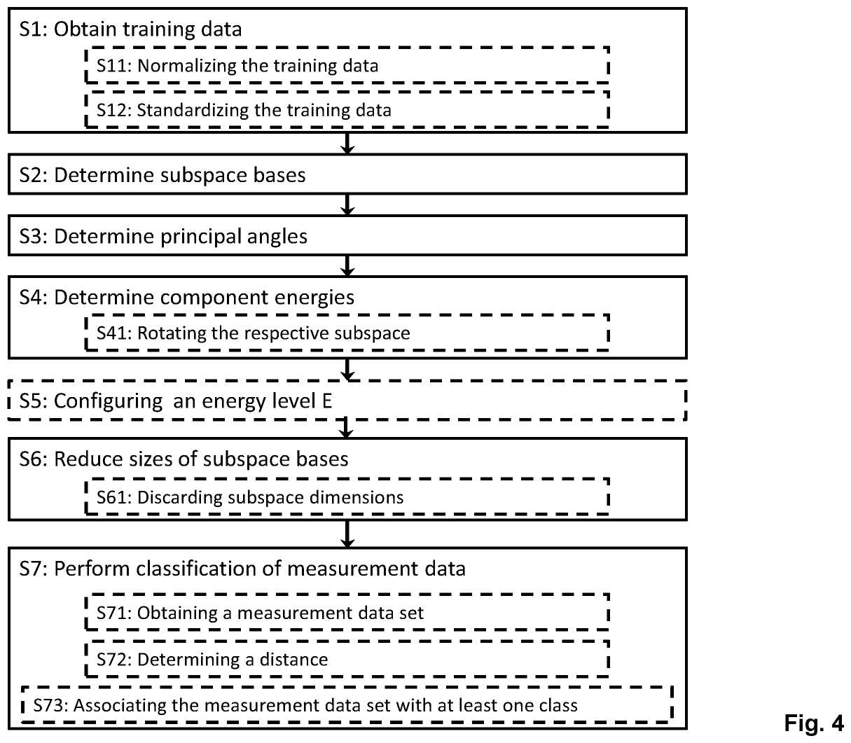

The diagnostic system is adapted to transmit one or more radio frequency signals into the body under test 103 from at least one of the antennas 105 in the system. The transmitted radio frequency signals are reflected and/or scattered by the internal object 100 due at least in part to the different dielectric properties of the internal object 100 compared to the body under test 103. The system is adapted to receive the reflected and/or scattered radio frequency signals at a receive antenna which may be the same antenna as used for transmitting or may be a different antenna. The system is also adapted to use a classification algorithm S1-S7, discussed in more detail below in connection to FIG. 4, to determine if the internal object 100 is present of not or determine if changes in a property of the internal object 100 has occurred. The classification algorithm S1-S7 illustrated in FIG. 4 is an example of a so-called machine learning algorithm, which is a self-learning algorithm. Before the algorithm can be used for detection of unknown targets a training phase is required and used to teach the algorithm to recognize the typical features associated with the presence of internal objects. In the training phase measurements are made on bodies under test 103 both without internal objects 100, and on bodies under test where internal objects 100 are present. In the training phase similar types of bodies under test 103 and internal objects 100 may be used that are later going to be measured and tested for the detection of internal objects.

For example, if the intended use of the system and method is to detect the presence of intracranial bleeding in a skull, patients with intracranial bleedings and healthy volunteers, without intracranial bleedings, are used in the training phase. The training phase can also in some cases be conducted on numerically simulated data or on measurements on phantom objects. It is appreciated that different training methods can be combined, i.e., be used as complements to each other. After the training phase, the diagnostic system with the classification algorithm S1-S7 can be used for detecting the presence of intracranial bleedings in patients, for example in an ambulance or at the pre-hospital field. The diagnostic system can also in some versions be used to detect and to monitor changes in intracranial bleedings in patients, i.e., to measure of the bleeding is improving or worsening.

One operation of the diagnostic system described herein is to detect presence of an internal object 100 or several internal objects 100. Another intended operation is to detect the internal object by means of analysing changes in the classification result over time of the received radio frequency signals. Yet another intended operation is to detect changes of properties, such as increase of size, position, shape, dielectric properties, etc., in an already detected internal object by analysing the differences in the classification results between radio frequency signals, such as microwave frequency radio signals, received at different times. Changes in the received radio frequency signals at different points in time are indicative of a change in properties of the internal object 100.

The detection of internal objects 100 is according to some aspects at least partly based on the self-learning classification algorithm S1-S7, which means that the classification algorithm comprised in the diagnostic system undergoes a training phase before it can be used for detecting internal objects. During the training phase, measurements should be made on subjects or samples with and without an internal object present. Based on this training data a classifier is built or configured and used in the analysis of measurement data.

For analysis, radio frequency signal scattering data are projected onto one or more, preferably two subspaces, or affine subspaces. In case there are two or more subspaces, one subspace may represent a situation when the internal object 100 is absent from the body under test 103, and one subspace may represent a situation where the internal object 100 is present within the body under test 103.

An orthogonal distance between two objects is the distance from one to the other, measured along a line that is perpendicular to one or both. Another way of explaining the same orthogonal distance is that it is defined as the shortest distance between two objects, such as a point and a line or hyperplane. According to one example one such distance measure is the Euclidean distance. Another example of orthogonal distance measure is the and the Mahalonobis distance. It is appreciated that several different definitions of distance can be used with similar effects, for instance: The distance can be measured as the length of the measurement's projection onto the subspace calculated from the origin, the length of the measurement's projection onto the affine subspace calculated from the mean of the training data for that class, the Manhattan distance from the measurement to the mean of the training data for that class, or it can be the angle between the measurement and the subspace.

According to an example, such an orthogonal distance from the measurement data to the projections onto two subspaces is computed. If the measurement data is closer to the subspace representing object presence, and the difference between the two distances is larger than a threshold, then the data is classified indicating that the internal object is present within the volume or body under test.

There is disclosed herein diagnostic methods, devices and systems for detecting presence of an object and/or for detecting changes in properties of an object comprised in an enclosing volume or body under test, where the object is associated with a dielectric property different from that of the volume or body under test. An integral part of the disclosed methods and systems is a classification method. An example of the disclosed classification method for two base spaces will be given here:

First, two base spaces are constructed with the use of training data. In some example embodiments, training data comprises radio frequency signals from test subject known to be healthy and from subjects who are known to have a brain haemorrhage. Each training measurement is represented by a two-dimensional array or similar structure with the first dimension representing the different wave frequencies, and the second dimension representing the transmission channels where the number of transmission channels equals the total number of combinations of sending and receiving antennas (a total of 36 combinations for 8 antennas). Each training measurement may also be formed into a vector where the first part of the vector represents the different wave frequencies for the first channel, the second part of the vector represent the wave frequencies of the second channel and continuing like this until all wave frequencies of all channels are included in the vector.

The entries of the array can be populated with S-parameter values or similar quantities representing propagation conditions for the given channel at the given frequency. Other examples of representations of the radio frequency scattering data could be z, y or h-parameters, reflection coefficients, insertion loss, etc. Other alternative representations calculated from measurements of transmitted and received radio frequency signals could also be used. For simplicity we refer to S-parameters in the following, but with the implicit understanding that other representations are equally applicable. At a given test frequency each element or S-parameter can be represented by a unitless complex number which represents magnitude and angle, i.e. amplitude and phase. The complex number may either be expressed in rectangular form or, more commonly, in polar form. The S-parameter magnitude may be expressed in linear form or logarithmic form. When expressed in logarithmic form, magnitude has the dimensionless unit of decibels, dB. The S-parameter angle is most frequently expressed in degrees but occasionally also in radians. The measurement of S-parameters is well known and will therefore not be discussed in more detail herein.

The vector of one or multiple measurements may be combined into a two-dimensional matrix, where each column represents the wave frequencies and channels of the first measurement, the second column the wave frequencies and channels of the second measurement, and so on. One may also combine in this way all measurements corresponding to measurements of healthy into such a matrix described above. This matrix may be referred to as the healthy data matrix. Similarly, all data that corresponds to the measurements on brain haemorrhages may be collected in to a matrix such as the one described above. This matrix may be called the bleeding data matrix.

According to another example only one subspace is constructed, e.g., a subspace basis for the measurements of the healthy subjects. By computing the orthogonal distance from a measurement to this single base space and classifying the measurement as bleeding if the distance is greater than a threshold and as healthy if less than the threshold.

A subspace spanning the healthy data matrix may be constructed using singular value decomposition. For example, a matrix X where each column of the matrix is one measurements vector, has the compact singular value decomposition

X=USV^{H}

Where U is an orthonormal matrix that spans the column space of X, S is a diagonal matrix with the singular values of X arranged in descending order on its diagonal, and V an orthonormal matrix that spans the row space of X. The columns of U are called the left singular vectors, and the columns of V the right singular vectors. In this case, the left singular vectors corresponding to the non-zero singular values represent the subspace which spans the healthy data matrix. This subspace may be referred to as the healthy subspace U_{H} and the diagonal matrix containing the corresponding singular values is denoted S_{H}. One may also construct an affine subspace of the healthy data matrix by computing the mean of all healthy measurements and subtracting this mean from all the healthy measurements. Similarly, we may construct a subspace spanning all the measurements of brain haemorrhages by the singular value decomposition of the bleeding data matrix. This subspace may be referred to as the bleeding subspace U_{B} and the diagonal matrix containing the corresponding singular values S_{B}. The columns of a matrix describing the basis for a space are denoted components. If the columns of U and V are computed only for the non-zero singular values, the singular value decomposition is denoted the “economy size” singular value decomposition.

One may then compute the principal angles between the two subspaces U_{H} and U_{B} by the singular value decomposition

U_{B}^{H}U_{H}=YCZ

where superscript H denotes Hermitian transpose, C is a diagonal matrix with the cosine of the smallest angles between the two subspaces on the diagonal elements sorted from the smallest angle to the largest angle, Y is a rotation matrix which rotates the coordinate axis of the bleeding subspace, Z is a rotation matrix which rotates the coordinate axis of the healthy subspace such that the first component of both subspaces have the cosine angle found in the first diagonal element of C to each other, the second component in both subspaces have the cosine angle found in the second diagonal element in C, and so on. The subspaces with rotated coordinates may be denoted Q_{H} for the healthy subspace and Q_{B} for the bleeding subspace,

Q_{H}=U_{H}Z,

Q_{B}=U_{B}Y.

The variance along the components of the rotated healthy subspace may be computed using Z and S_{H}. The variance w_{H,i} along the ith component of Q_{H} is

w_{H,i}=zᵢ^{H}S_{H}²zᵢ

where zᵢ is the ith column of Y. Similarly, the variance w_{B,i} of the ith component of Q_{B} is

w_{B,i}=yᵢ^{H}S_{B}²yᵢ

where yᵢ is the ith column of Y.

Some components of U_{H}, U_{B}, Q_{H}, and Q_{B} may be discarded to remove noise, reduce the dimension of the subspace, and for regularization of the classifier. Here the term component is referring to one column of U_{H}, U_{B}, Q_{H}, and Q_{B}. When applying radio frequency signal scattering data towards the use of diagnostics, it should be appreciated that the number of dimensions of the measured data may exceed the number of training samples available. This problem is alleviated by the techniques proposed herein, due to the discarding of subspace components or dimensions.

One method to select which components to remove is to remove the singular vectors that correspond to the smallest singular values, this is in principle how it is done in PCA, principle component analysis. Another method is to remove the components in Q_{H} and Q_{B} that corresponds to the smallest principal angles, as is also known from the literature. A third method is to not discard any components.

According to the present disclosure the method to remove singular vectors is to combine the variance along the components of the rotated subspaces with the principal angles between the two subspaces. Components which have low principal angle and low variance are discarded, as these are judged to contain both little information of the data itself and little discriminatory information. Components of the two subspaces with low variance and small principal angles are thus discarded from the bases. One example of how to determine the number of components to discard is to count the total variance and not remove more than a certain amount of the total variance. One may, for example, truncate the subspace such that at least 95% remains. One may also set a fixed limit on the number of components to discard or use any combination of compatible criteria.

When components of the two subspaces have been removed, a measurement can be classified by computing the distance from the measurement to the subspace. The distance can be the orthogonal distance from the measurement to the subspace, the length of the measurement's projection onto the subspace calculated from the origin, the length of the measurement's projection onto the affine subspace calculated from the mean of the training data for that class, the Manhattan distance from the measurement to the mean of the training data for that class, or it can be the angle between the measurement and the subspace. According to the present disclosure, the classification may be done by computing the difference between the distance from a measurement to the healthy subspace and the distance to the bleeding subspace. A combination of the distances can also be used with any type of classifier, such as Quadratic discriminant analysis (QDA), decision trees or forests, Support Vector Machine (SVM), K-Nearest Neighbours (KNN), neural network, or a subspace classifier. It is preferred that the algorithm outputs a single real number, a real-valued scalar, but adaptations to other output formats are possible.

If the difference between the two distances is greater than a threshold, the measurement is classified as bleeding, and if the distance difference or the real-valued scalar is lower than the threshold, the measurement is classified as healthy.

FIG. 1 illustrates an example of a measurement device 10 with one antenna for detecting an internal object 100 in a body under test 103. If only one antenna is used only reflection measurements can be made. The internal object 100 and the body under test 103 are not parts of the system. The body under test 103 may be a head, a brain, an abdomen, a thorax, a leg or any other body under test part of a human, an animal, or it may be any other form of biological tissue such as for example a tree or wood. The body under test 103 may also be non-living tissue, and of non-biological origin, such as, but not limited to plastics, etc. The body under test 103 may also be referred to as a dielectric medium, an object under investigation, a larger object, etc. The internal object 100 may also be referred to as an immersed object, a dielectric target, etc. The internal object 100 may be in the form of solid, semisolid, liquid or gas. The internal object 100 may be referred to as an immersed object in a larger object or body under test 103. The internal object 100 may also be referred to as a dielectric target, with certain properties, such as size, shape, position, dielectric parameters, etc. that is immersed inside another dielectric medium, i.e. the body under test 103. The internal object 100 may be a bleeding, a clot, an edema, a nail, a twig etc. Note that FIG. 1 only illustrates one internal object 100, but any number of internal objects 100 may be present in the body under test 103, including no objects at all. One internal object 100 is shown for the sake of simplicity.

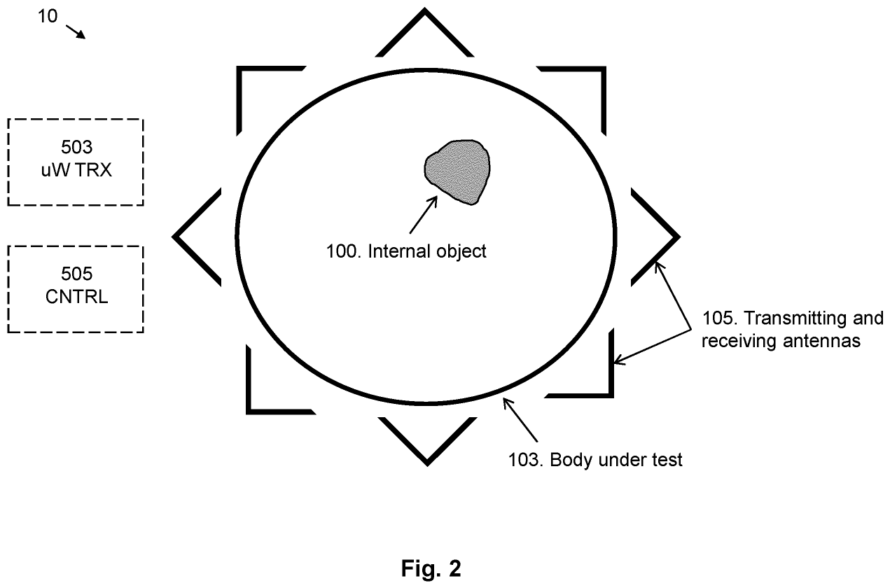

FIG. 2 illustrates an example of a measurement device 10 comprising eight antennas for detecting an internal object 100 in a body under test 103. If two or more antennas are used both reflection and transmission measurements can be made and used for classification. In principle, all antenna combinations can be used for measurements, but for different reasons one might want to use only a subset of all possible combinations. Therefore, according to some aspects, before using the system for a particular application it should be decided which antenna combinations to use. In the following description, a set of measurements including all the desired antennas is denoted a “full measurement set”. FIG. 2 also schematically shows the microwave transceiver 503 and the control unit 505 that was briefly mentioned above.

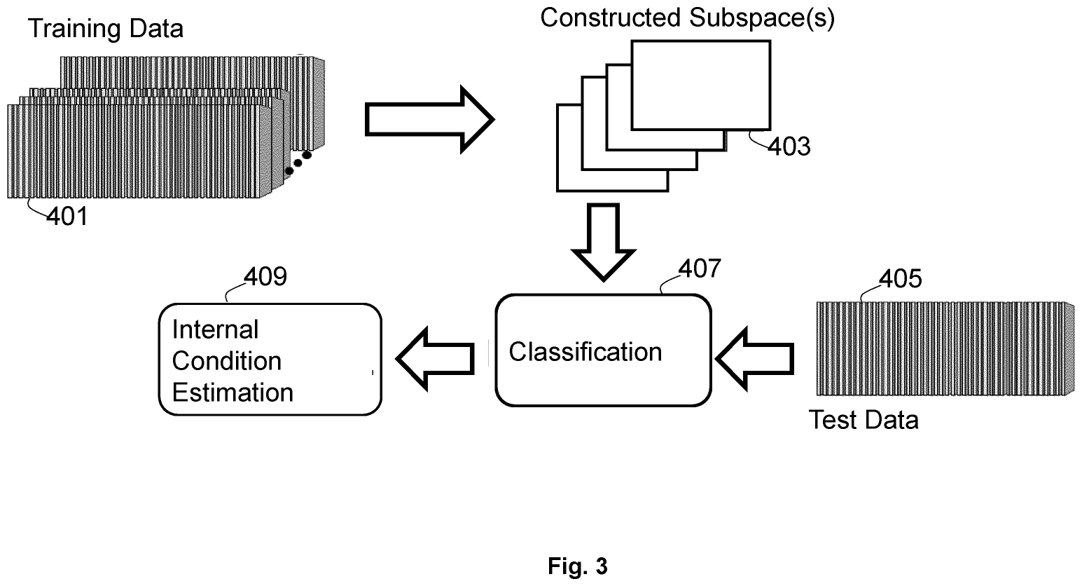

FIG. 3 illustrates an example of how the self-learning classification algorithm described herein is implemented and how the detection phase is depending on the training phase. FIG. 3 is an example of how the processing circuitry in the control unit 505 may operate. The first step is to perform radio frequency signal measurements to collect training data 401 from samples or subjects with known conditions. Once enough training data 401 has been collected, next step is to use the training data 401 to form subspaces 403. This is referred to as the training of the classifier, and several aspects related to the training and tuning of the classifier is disclosed herein. The subspaces 403 contain the information needed to perform the classification 407 of test data 405, measured on a body under test 103 and in which the task is to determine if an internal object 100 is present or not. From the classification, an internal condition estimation 409 is given. This result can for example be calculated in the analyser 505 and presented on the display unit 507.

FIG. 4 illustrates with a flow chart an example of the different steps of the training and the classification phases. First step S1 is to collect training data 401, next step S2 is to determine the subspace bases 403 associated with the different classes, next step S3 is to determine the principal angles between all pairs of components within the subspace bases, next step S4 is to determine the energy levels of each component in the subspaces, an optional next step comprises configuring S5 an energy level E used in reducing the dimension subspace for each class, next step S6 is to reduce the sizes of the subspace bases based on the component energies and principal angles. All these steps can be made a priori to the actual step of classifying 407 test data 405 originating from measurements of a body under test 103. The classification is made by executing the step S7. The resulting subspaces 403 obtained after step S6 can be stored in a computer memory and used for classifying 407 test data 405.

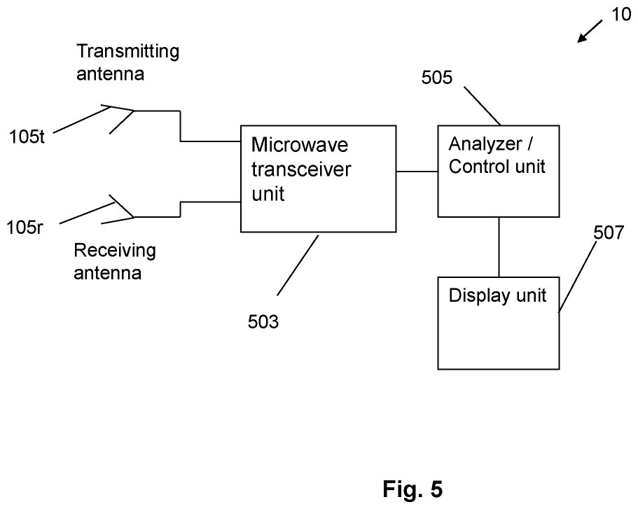

Some components of the measurement devices 10 and systems described herein are depicted in FIG. 5 and consists of at least one transmitting antenna 105t and at least one receiving antenna 105r. When using the reference number 105 without the letters t or r, it refers to any of the transmitting and receiving antennas. It should be noted that these two antennas 105 may be combined in one antenna and in such case a directing mechanism (not shown) may be arranged in the path between the antenna 105 and the radio frequency transceiver inside the transceiver or as an external device. The combined transmitting and receiving antenna 105 may be referred to as a transceiver. The directing mechanism may be used in order to not transmit directly into a receiving unit in the transceiver possibly saturating the input electronics. A combination of the radio frequency transmitting/receiving unit 503 and the analyzer 505 may have a direction dependent component, e.g. the directing mechanism, which controls the transmitted and received signal in different directions. This may take place at the same time, i.e. transmission and reception may take place simultaneously. The directing mechanism may also be referred to as a switching mechanism. The transceiver may comprise two separate units, a transmitting unit and a receiving unit, or it may be built into one single unit with electronics for each function built into the single unit. The antennas are connected to a radio frequency transmitting/receiving unit 503 that is adapted to transmit and to receive radio frequency signals to and from the antennas 105. The system may further comprise an analyser 505 that is arranged to control a display unit 507. The display unit 507 is adapted to display the analysed result of the signals e.g. on a screen. Signal analysis may be performed at another location by sending, through a network connection or using storage devices, measured signals to another analysis device, e.g. a central server or central computational device for post analysis and/or for storing of measured signals in a central storage facility. This analysis device may be the same as the analyser 505 in FIG. 1 or a different analyser. The training phase of the classifier as well as the execution of the detection algorithm in order to produce a detection result based on the radio frequency signal measurements is performed on the analyser 505, the results are then presented on the display unit 507.

An example of a configuration of a system for detecting internal objects 100 inside a body under test 103 is given below:
at least one antenna operating as transmitter 105t and one antenna operating as receiver 105r where in one embodiment the transmitting and receiving antenna can be the same;
the antennas being positioned in close proximity to a body under test 103;
the antennas 105 used for sending and receiving radio frequency signals towards and receiving scattering signals from the body under test 103, the antennas 105 further being connected to a radio frequency transmitting/receiving unit 503 adapted to send and receive radio frequency signals the transmitting/receiving unit 503 being connected to an analyser 505 that is adapted to analyse the measured radio frequency signal scattering data;
the analyser 505 being configured to analyse the microwave signals in order to determine if an internal object 100 is present in body under test 103;
the detection of the presence of the internal object 100 is made in a classification algorithm S1-S7 that bases its detection on a comparison measurement test data 405 with a database containing a set of training data 410;
a display unit 507 configured to present the detection result.

In one example embodiment the system is further configured to analyse the measured test data using a classifier and a set of training data stored in a database; the training data collected from several cases with the internal object present and known to be in configurations representative of what can be expected in the desired detection scenario, and also for several cases where the internal object was known not to be present and where the body under test was known to be in representative configurations of the detection scenario.

In one example embodiment the system is further configured to use training data collected for cases where the internal object was present and for cases when the internal object was not present; where data from the radio frequency signal measurements are projected onto two subspaces, one representing a situation when the internal object is absent within the body under test, and one where the internal object is present within the body under test.

In one example embodiment the system is further configured such that training data was collected for one class, either with the internal object present or not, where data from the radio frequency signal measurements are projected onto one subspace, representing the situation when the internal object is present or not.

In one example embodiment the system is further configured such that the classification is made by executing the algorithm according to the steps S1-S7.

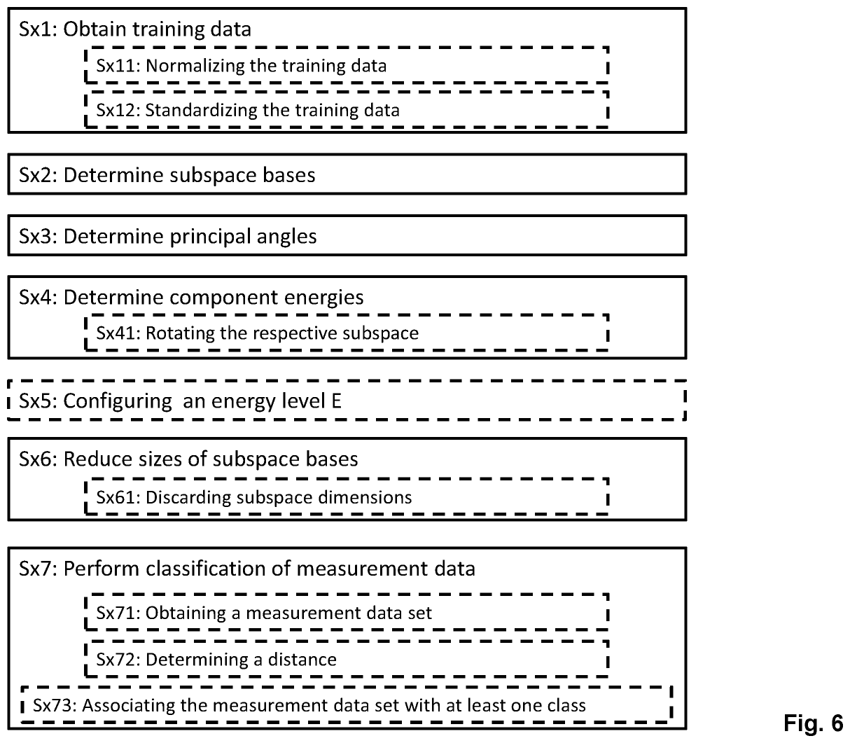

FIG. 6 illustrates modules Sx1-Sx7 which may be comprised in processing circuitry of a control unit 505.

Below a detailed description is given of an example for how the training data 401 is used to determine subspaces 403 and how the classification 407 of measured test data 405 is made by executing steps according to S1-S7.

Arrange each sample of training data 401, e.g. radio frequency signal scattering data, into a column vector, i.e. vectorise each measurement. The measurement data can be either real or complex. For each of the two classes, w⁽¹⁾ and w⁽²⁾, e.g. healthy and bleeding patients, construct a matrix where each column is one vectorised data sample from that class. These matrices are referred to herein as “measurement matrices”, or, when considering only one matrix, the “measurement matrix”.

Compute one basis for the range space and the singular values of each of the measurement matrices by means of the “economy size” singular value decomposition. We call these bases the “subspace bases”, or, when considering only one, the “subspace basis”.

Compute the principal angles between the subspace bases by means of the singular value decomposition. Compute the energy in the components associated to the principal angles. Combine the principal angles together with their associated component energy. Remove components with small combined component energy and principal angle scores, i.e. remove the components with the smallest component energy and principal angle, from the subspace bases. Different methods of combining the principal angles and the component energies are possible.

The two reduced subspace bases are used to predict the class belonging of new data samples.

Note that the proposed techniques can handle both non-centred data and centred data. Centred data means that the mean of each class has been subtracted from each measurement of that class. During prediction, the class mean of respective class is subtracted from the data sample before it is projected onto the corresponding subspace basis.

The classifier bases its function on three factors: the principal angles, PA, and the component energies of each of the two classes, w⁽¹⁾ and w⁽²⁾.

In the following, class belonging is written as superscript (c) where c=1 or c=2. Thus, the data matrix for class 1 is written X⁽¹⁾. If class belonging is written (c), the same operation is performed on both classes independently. It is also assumed that each column in matrix X^{(c)} corresponds to one measurement.

The proposed method comprises obtaining S1 training data.

The proposed classifier supports normalization and standardization of the training data 401. Normalization means that the (row-wise) mean of each class is estimated and subtracted from the data of respective class, i.e.,

$${\tilde{X}}^{( c )} = {X}^{( c )} - 〈 {X}^{( c )} 〉 .$$

$$Here$$

$$〈 {X}^{( c )} 〉 = \frac{1}{{N}_{c}} {\sum }_{i = 1}^{{N}_{c}} {x}_{i}^{( c )}$$

Where xᵢ^{(c) is the i-th sample of class c and N}_{c} the number of samples in this class.

Standardization means that the row-wise class-specific mean is subtracted from the class data and then the difference is divided by the class-specific (row-wise) variance, i.e.

$${\tilde{X}}^{( c )} = \frac{{X}^{( c )} - 〈 {X}^{( c )} 〉}{{σ ( {X}^{( c )} )}^{2}} ,$$

where σ(X^{(c)}) is the row-wise standard deviation of class c, i.e.

$${σ ( {X}^{( c )} )}^{2} = \frac{1}{{N}_{c} - 1} {\sum }_{i = 1}^{N c} ( {x}_{i}^{( c )} - 〈 {X}^{( c )} 〉 ) {( {x}_{i}^{( c )} - 〈 {X}^{( c )} 〉 )}^{H}$$

In the cases when mean normalization or standardization is used, the mean and variance of the classes are saved and used when evaluating the classifier on test data.

The proposed classifier has one hyperparameter, the energy E that must remain in the classes after component removal. The optimal value of this hyperparameter can be found by tuning. Tuning may be performed using cross-validation: First, put aside part of the training set. Train a classifier with one set of hyperparameters on the remainder of the training set. Evaluate the trained classifier's performance on the held-out part of the training set. Set aside a new part of the training set, train on the remainder and evaluate on the held-out part. Continue this practice until all samples of the training set have been in the held-out set once. Compute the overall performance from all the held-out sets. Select a new hyperparameter setting and redo the training and hold-out procedure. Continue until all hyperparameter settings have been used. The hyperparameter setting with the highest overall performance is chosen as the optimal setting.

After any normalization or standardization of the training data, the classifier computes a subspace basis U^{(c)} and the singular values S^{(c)} of each class by means of the, economy-sized, singular value decomposition

X^{(c)}=U^{(c)}S^{(c)}V^{(c)}^{^{H}},

where S^{(c)}=diag(σ₁^{(c)},σ₂^{(c)}, . . . , σ_{N}_{^{(c)}}^{(c)}) is a diagonal matrix with the singular values on the main diagonal and N^{(c)} is the number of measurements from class c. Note that V^{(c)} is not necessary for the function of the classifier. The superscript H again denotes Hermitian transpose.

Thus, the disclosed method comprises determining S2 a subspace base for each class out of the one or more classes based on the training data.

The principal angles between the two subspaces are computed via a second singular value decomposition

U⁽¹⁾^{^{H}}U⁽²⁾=Y⁽¹⁾CY⁽²⁾^{^{H}},

where Y⁽¹⁾ and Y⁽²⁾ are square unitary matrices,

C=diag(cos θ₁, cos θ₂, . . . , cos θ_{N}), θᵢ is the ith principal angle, and

N=max(N⁽¹⁾,N⁽²⁾).

The determination of matrix C is an example of determining S3 principal angles between each pair of subspace bases.

We now define a (row-)vector of the principal angles as

PA=[cos θ₁,cos θ₂, . . . ,cos θ_{N}]^{T}

By rotating the class bases U⁽¹⁾ and U⁽²⁾ using the rotation matrices Y⁽¹⁾ and Y⁽²⁾ according to

\tilde{Q}⁽¹⁾=U⁽¹⁾Y⁽¹⁾

\tilde{Q}⁽²⁾=U⁽²⁾Y⁽²⁾,

we have the bases \tilde{Q}⁽¹⁾ and \tilde{Q}⁽²⁾ of the classes 1 and 2 in coordinate systems such that \tilde{q}₁⁽¹⁾ and \tilde{q}₁⁽²⁾, i.e. the first component (column) of \tilde{Q}⁽¹⁾ and \tilde{Q}⁽²⁾ respectively, have the angle θ₁ to each other. The angles between the remaining pairs of components follow the same scheme. In case one class contain n more components than the other, the last n elements in PA equals zero. Parallel components show up in PA as ones.

To determine component energies w⁽¹⁾ and w⁽²⁾ we use the singular values S^{(c)} and the rotation matrices Y^{(c)}, namely, wᵢ^{(c)}, the ith component energy in class c, are calculated as

wᵢ^{(c)}=yᵢ^{(c)}^{^{H}}S^{(c)}^{²}yᵢ^{(c)},

where yᵢ^{(c)} is the ith component (column) in Y^{(c)}. The vector w⁽¹⁾ and w⁽²⁾ are, simply,

w^{(c)}=[w₁^{(c)},w₂^{(c)}, . . . ,w_{N}_{^{(c)}}^{(c)}]^{T}.

This is an example of determining S4 a component energy for each dimension in each subspace.

For simplicity, only the component selection of the first class, c=1, is described in the following. The same component selection method may be applied to other classes. First, for each component the following product is computed

$${f}_{i}^{( 1 )} ( P {A}_{i} , {w}_{i} ) = {( 1 - P {A}_{i} )}^{α} ⊙ {( \frac{{w}_{i}^{{( 1 )}^{2}}}{{w}_{tot}^{{( 1 )}^{2}}} )}^{β} ,$$

where the exponents are elementwise, α and β are real-valued constants, ⊙ is the element-wise multiplication operation (.* in matlab), and

$${w}_{tot}^{{( 1 )}^{2}} = {\sum }_{i = 1}^{{N}^{( 1 )}} {w}_{i}^{{( 1 )}^{2}}$$

is the total energy in class c and is used to normalized the energy components between zero and one. This function become large when PAᵢ is small, i.e. large angle, and wᵢ⁽¹⁾ is large. The principal angle term is by construction bounded between zero and one. The two factors α and β can be varied to change the relative importance of the principal angles and the component energies.

When ƒᵢ⁽¹⁾ is computed for all i, the list ƒ⁽¹⁾=[ƒ₁⁽¹⁾, ƒ₂⁽¹⁾, . . . ƒ_{N}_{⁽¹⁾}⁽¹⁾]^{T} is defined. This list is then sorted from the largest to the smallest. The sort order is given in the vector

j=sort([ƒ₁⁽¹⁾,ƒ₂⁽¹⁾, . . . ƒ_{N}_{⁽¹⁾}⁽¹⁾]^{T},descending),

i.e. j₁ is the index of the largest element in ƒ⁽¹⁾, or j₁=ᵢmax ƒᵢ⁽¹⁾. The second index j₂ is the index of the second largest element in ƒ⁽¹⁾, and so on.

The next step is to create the cumulative component energy list

$${w}_{c u m}^{{( 1 )}^{2}} = {[ {w}_{{j}_{1}}^{{( 1 )}^{2}} , {w}_{{j}_{1}}^{{( 1 )}^{2}} + {w}_{{j}_{2}}^{{( 1 )}^{2}} , … , {\sum }_{i = 1}^{{N}^{( 1 )} - 1} {w}_{{j}_{i}}^{{( 1 )}^{2}} , {\sum }_{i = 1}^{{N}^{( 1 )}} {w}_{{j}_{i}}^{{( 1 )}^{2}} ]}^{T} .$$

This is the cumulative sum of the component energies, which we use to ensure that we retain a certain amount of total energy in the classes after discarding components. For each class, we take the hyperparameter E and find the first element, we index it k here, in w_{cum}⁽¹⁾^{²} that satisfies

$$\frac{{w}_{cum , k}^{{( 1 )}^{2}}}{{w}_{tot}^{{( 1 )}^{2}}} \geq E ,$$

i.e. k is the number of components needed for the cumulative energy to equal or exceed the energy minimum E.

The final step is to discard components from the subspace bases, and create the final bases as

Q⁽¹⁾=[\tilde{q}ⱼ_{₁}⁽¹⁾,\tilde{q}ⱼ_{₂}⁽¹⁾, . . . ,\tilde{q}ⱼ_{ₖ}⁽¹⁾],

where \tilde{q}ⱼ_{ₖ}⁽¹⁾ is the jₖth component of the basis \tilde{Q}⁽¹⁾.

Now, we have created an orthonormal subspace base Q⁽¹⁾ that retains a given amount of total energy composed solely of the most important components of the basis \tilde{Q}⁽¹⁾, according to the sorting function ƒᵢ⁽¹⁾(PAᵢ,wᵢ). Thus, there has been provided an example of determining S6 a reduced dimension subspace for each class by discarding subspace dimensions based on respective principal angle and component energy.

The sorting procedure is repeated for the class c=2 to create the subspace basis Q⁽²⁾.

When the two class bases Q⁽¹⁾ and Q⁽²⁾ have been created, the class associated with a new, previously unseen, data point can be predicted. The class of an unknown data point is predicted by computing a number that indicates which of the two classes the data point is closest to. The definition of “closest to” may be defined in different ways depending on application, as discussed above in connection to discussing different types of distances, e.g., orthogonal distance. For subspace-based classifiers, it is convenient to use a distance computed from the unknown data point to each of the classes. This distance can be the (closest) distance from the data point to each of the two classes, the length of the projection of the data point onto each class, the Manhattan distance from the class mean to the data point, i.e. the length of the projection plus the distance from the data point to the class, etc.

The prediction entails computing the difference between the two distances, and selecting the class as

$${d}^{( 2 )} - {d}^{( 1 )} \begin{matrix}c = 1 \\ \begin{matrix}> \\ <\end{matrix} \\ c = 2\end{matrix} β ,$$

which should be read as “If d₁+β is shorter than d₂, the data point is predicted to belong to class 1, and vice versa.”. The factor β is a tunable constant which can be set to control the specificity and sensitivity of the classifier and is swept from −∞ to ∞ when computing the AUC.

Mathematically, the (squared) length of the projection of a data point x onto the class c base matrix Q^{(c)} and class mean m^{(c)} is

dₚᵣₒⱼ^{(c)}^{²}=∥Q^{(c)}(Q^{(c)}^{^{H}}Q^{(c)})⁻¹Q^{(c)}^{^{H}}(x−m^{(c)})∥₂²=(x−m^{(c)})^{H}Q^{(c)}Q^{(c)}^{^{H}}(x−m^{(c)}),

due to Q^{(c)} being a unitary matrix. It is not necessary to compute the square root of the equation above, since this will not change the which of d⁽¹⁾ or d⁽²⁾ is the largest.

The (squared) distance from a data point x to the class c base matrix Q^{(c)} and class mean m^{(c)} is

d_{dist}^{(c)}^{²}=∥(I−Q^{(c)}(Q^{(c)}^{^{H}}Q^{(c)})⁻¹Q^{(c)}^{^{H}}(x−m^{(c)})∥₂²=(x−m^{(c)})^{H}Q^{(c)}Q^{(c)}^{^{H}}(x−m^{(c)}).

As in the case of the length of the projection, it is ok to use the squared distance when predicting.

The (non-squared) Manhattan distance from a data point x to the mean m^{(c)} of class c with basis Q^{(c)} is

d_{Man}^{(c)}=dₚᵣₒⱼ^{(c)}+d_{dist}^{(c)}.

Note that we cannot use the squared length of the projection or distance here. Using the squared length of projection and distance computes the distance from the mean of the class to the data point,

dₚᵣₒⱼ^{(c)}^{²}+d_{dist}^{(c)}^{²}=(x−m^{(c)})^{H}(x−m^{(c)}),

and does not contain any information of the subspace base Q^{(c)}.

Subspace-based classifiers are used extensively in the field of machine learning. One-class classifiers aim to predict whether a measurement of unknown origin, belongs to the class or is a so-called outlier or anomaly. This can be done in multiple ways, e.g. thresholding the distance from the measurement to its orthogonal or oblique projection on the subspace, the angle between the measurement and the subspace, etc., or a combination of multiple metrics. Two-class or multi-class classifiers, where the proposed classifier belongs to the group of two-class classifiers, on the other hand, computes a distance (or angle, etc.) metric to each of the classes. The metric can, again, be the distance to or the length of an orthogonal or oblique projection, an angle to the subspace, etc., or a combination of multiple metrics.

There is also a possibility to truncate the subspaces in order to improve or introduce separation between the subspaces that describe the different classes, or to regularize the classifier. For instance, in “A unified subspace classification framework developed for diagnostic system using microwave signal,” 21st European Signal Processing Conference (EUSIPCO 2013), Marrakech, 2013, pp. 1-5, Yinan Yu and T. McKelvey describes a method comprising removing parts of the subspaces that have small principal angles. Many subspace-based classifiers use the principal components of the data constituting the different classes in order to reduce the dimensionality of the subspaces.

In contrast to prior art, the proposed classifier does not only use principal components, eigenvalues, singular values, or principal angles to truncate the subspace, but a combination of these. The principal components carry information on which constituents of the subspaces that carry the majority of the information for each individual class, while the principal angles contain information on the similarities between the subspaces. Thus, by combining the knowledge from the two, we can construct subspaces that carry more information while achieving high separation between the class subspaces. Truncation of the subspaces are then done in a way that maximizes the principal angles and the variance explained by the components of the classes simultaneously. Further, the proposed classifier is different from performing dimensionality reduction with principal component analysis followed by empirical subspace intersection removal as described in “A unified subspace classification framework developed for diagnostic system using microwave signal,” 21st European Signal Processing Conference (EUSIPCO 2013), Marrakech, 2013, pp. 1-5, Yinan Yu and T. McKelvey as no information is lost until in the very last step. Performing the truncation using principal components before the principal angles are computed, or vice versa, causes unnecessary information loss.

FIG. 7 schematically illustrates, in terms of a number of functional units, the components of the control unit 700 discussed above. This control unit 700 may be comprised in a measurement system 10, e.g., in the form of a VMM unit. Processing circuitry 710 is provided using any combination of one or more of a suitable central processing unit CPU, multiprocessor, microcontroller, digital signal processor DSP, etc., capable of executing software instructions stored in a computer program product, e.g. in the form of a storage medium 730. The processing circuitry 710 may further be provided as at least one application specific integrated circuit ASIC, or field programmable gate array FPGA.

Particularly, the processing circuitry 710 is configured to cause the control unit 700 to perform a set of operations, or steps, such as the methods discussed in connection to FIG. 10. For example, the storage medium 730 may store the set of operations, and the processing circuitry 710 may be configured to retrieve the set of operations from the storage medium 730 to cause the control unit 700 to perform the set of operations. The set of operations may be provided as a set of executable instructions. Thus, the processing circuitry 710 is thereby arranged to execute methods as herein disclosed.

The storage medium 730 may also comprise persistent storage, which, for example, can be any single one or combination of magnetic memory, optical memory, solid state memory or even remotely mounted memory.

The control unit 700 may further comprise an interface 720 for communications with at least one external device. As such the interface 720 may comprise one or more transmitters and receivers, comprising analogue and digital components and a suitable number of ports for wireline or wireless communication.

The processing circuitry 710 controls the general operation of the control unit 700, e.g., by sending data and control signals to the interface 720 and the storage medium 730, by receiving data and reports from the interface 720, and by retrieving data and instructions from the storage medium 730. Other components, as well as the related functionality, of the control node are omitted in order not to obscure the concepts presented herein.

FIG. 8 illustrates a computer readable medium 810 carrying a computer program comprising program code means 820 for performing the methods discussed herein when said program product is run on a computer. The computer readable medium and the code means may together form a computer program product 800.

1. A measurement device comprising at least one transmitting antenna, at least one receiving antenna, a microwave transceiver unit connected to the at least one transmitting antenna and to the at least one receiving antenna, and a control unit connected to the microwave transceiver unit, the control unit comprising processing circuitry arranged to classify measurement data obtained via the microwave transceiver unit into one or more pre-determined classes, the processing circuitry comprising;
an obtaining module arranged to obtain training data,
a first determining module configured to determine a subspace base for each class out of the one or more classes based on the training data,
a second determining module configured to determine principal angles between each pair of subspace bases,
a third determining module configured to determine a component energy for each dimension in each subspace corresponding to the principal angles
a fourth determining module configured to determine a reduced dimension subspace for each class by discarding subspace dimensions based on respective principal angle and component energy, and
a classifying module arranged to classify the measurement data into the one or more classes based on the reduced dimension subspaces.

2. The measurement device according to claim 1, wherein the control unit is arranged to determine an energy level E, wherein the fourth determining module is arranged to determine the reduced dimension subspace for each class by discarding subspace dimensions while maintaining an energy level per subspace above the configured energy level E.

3. The measurement device according to claim 1, wherein the third determining module is configured to determine the component energy by rotating the respective subspace to correspond to the principal angles.

4. The measurement device according to claim 1, wherein the obtaining module is configured to normalize the obtained training data.

5. The measurement device according to claim 1, wherein the obtaining module is configured to obtain the training data as standardized training data.

6. The measurement device according to claim 1, wherein the classifying module is configured to perform the classification by
obtaining a measurement data set,
determining a distance between the measurement data set and at least one of the reduced dimension subspaces corresponding to the one or more classes, and
associating the measurement data set with at least one class based on the determined distance.

7. The measurement device according to claim 1, wherein the one or more classes comprises a class corresponding to non-defect wood, and a class corresponding to defect wood.

8. The measurement device according to claim 1, wherein the one or more classes comprises a class corresponding to healthy patients, and a class corresponding to patients with brain haemorrhage or brain stroke.

9. A method for classifying measurement data into one or more classes, the method comprising;
obtaining training data,
determining a subspace base for each class out of the one or more classes based on the training data,
determining principal angles between each pair of subspace bases,
determining a component energy for each dimension in each subspace corresponding to the principal angles
determining a reduced dimension subspace for each class by discarding subspace dimensions based on respective principal angle and component energy, and
classifying the measurement data into the one or more classes based on the reduced dimension subspaces.

10. The method according to claim 9 wherein the one or more classes comprises a class corresponding to non-defect wood, and a class corresponding to defect wood.

11. The measurement device according to claim 2, wherein the third determining module is configured to determine the component energy by rotating the respective subspace to correspond to the principal angles.

12. The measurement device according to claim 2, wherein the obtaining module is configured to normalize the obtained training data.

13. The measurement device according to claim 3, wherein the obtaining module is configured to normalize the obtained training data.

14. The measurement device according to claim 2, wherein the obtaining module is configured to obtain the training data as standardized training data.

15. The measurement device according to claim 3, wherein the obtaining module is configured to obtain the training data as standardized training data.

16. The measurement device according to claim 4, wherein the obtaining module is configured to obtain the training data as standardized training data.

17. The measurement device according to claim 2, wherein the classifying module is configured to perform the classification by
obtaining a measurement data set,
determining a distance between the measurement data set and at least one of the reduced dimension subspaces corresponding to the one or more classes, and
associating the measurement data set with at least one class based on the determined distance.

18. The measurement device according to claim 3, wherein the classifying module is configured to perform the classification by
obtaining a measurement data set,
determining a distance between the measurement data set and at least one of the reduced dimension subspaces corresponding to the one or more classes, and
associating the measurement data set with at least one class based on the determined distance.

19. The measurement device according to claim 4, wherein the classifying module is configured to perform the classification by
obtaining a measurement data set,
determining a distance between the measurement data set and at least one of the reduced dimension subspaces corresponding to the one or more classes, and
associating the measurement data set with at least one class based on the determined distance.

20. The measurement device according to claim 5, wherein the classifying module is configured to perform the classification by
obtaining a measurement data set,
determining a distance between the measurement data set and at least one of the reduced dimension subspaces corresponding to the one or more classes, and
associating the measurement data set with at least one class based on the determined distance.
